(19) United States
(12) Patent Application Publication
(10) Pub. No.: US 2007/0235263 A1
(43) Pub. Date: Oct. 11, 2007

(54) Method for displaying an array of packages

(76) Inventors: Dennis Xavier Legault, Amelia, OH (US); Sundar G. Raman, Cincinnati, OH (US); Marchoe Lynn Dill, Township, OH (US); Jacques Dickran Hagopian, Cincinnati, OH (US); Jennifer Lyn Pustinger, Terrace Park, OH (US); Kristine Gail Noschang, Cincinnati, OH (US); Surya Narayana Menon, Mason, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/263,396

(22) Filed: Oct. 31, 2005

(51) Int. Cl. A61F 13/15

(52) U.S. Cl. 186/52

(57) ABSTRACT

A method for displaying an array of packages including providing at least one first package including a product therein. The first package has at least a first display face and a first display face area that includes first indicia having a first area and representing the name of the product or the product manufacturer and second indicia having a second area including an equity building advertising message for the product in the package. The second area of the second indicia is greater than the first area of the first indicia on the first display face of the first package. The method also includes providing at least a second package including a product therein. The second display face includes the first indicia of the first package and does not include the second indicia of the first package.

### FIELD OF THE INVENTION

The present invention relates to an improved method for displaying an array of packages.

### BACKGROUND OF THE INVENTION

Consumer and other products are displayed and advertised in many different ways. Over the years, there have been numerous attempts to maximize consumer awareness of such products in order to increase sales of the products. One way to increase consumer awareness is to advertise the product. This can, and has, been done by many different means, including, but not limited to television, radio, print and other mass media, coupons, in-store advertising, direct mailing and e-mailing, catalogs and web site links and advertisements.

In other instances, the products may be placed in stores or shown on websites in ways to increase the likelihood that a consumer will see the product and purchase it. For example, retailers may place a particular product in a particular location of the store (e.g. the end of the aisle or along the check-out line) in an attempt to increase the product's visibility to the consumer.

Yet another way to entice consumers to purchase and try a particular product may be to provide the product with unique packaging. Such packaging may be unique in terms of its shape, functionality, appearance or any other aspect that may attract a potential buyer of the product. One proven method to gain and sustain product sales is to use unique identifiers on the product packaging, such as trademarks and/or trade dress.

However, despite the numerous and continuing attempts to make consumers aware of products and to maintain and/or increase sales of such products, there still exists a need to provide products with an improved method for advertising such products and/or the benefits of such products.

There also exists a need to provide an improved method for arranging a number of the same products or an array of similar products on a shelf or other storage device.

There also exists a need to provide an improved method for advertising a product or array of products on a website.

There also exists a need to provide an improved package including advertising the benefits of the product provided therein.

### SUMMARY OF THE INVENTION

In order to address the above-mentioned needs in the industry, the present invention provides a method for displaying an array of packages including providing at least one first package including a product therein. The first package has at least a first display face and a first non-display face. The first display face is intended to be displayed to a consumer when the package is displayed for sale. The first display face has a first display face area and includes first indicia having a first area and representing the name of the product or the product manufacturer and second indicia having a second area including an equity building advertising message for the product in the package. The second area of the second indicia is greater than the first area of the first indicia on the first display face of the first package. The method also includes providing at least a second package including a product therein. The second package has a second display face that is intended to be displayed to a consumer when the package is displayed for sale. The second display face includes the first indicia of the first package and does not include the second indicia of the first package.

### BRIEF DESCRIPTION OF THE DRAWINGS

FIG. 1 is an isometric view of a prior art package with a section cut away to show the product therein.

FIG. 2 is an isometric view of one example of an embodiment of the present invention with a section cut away to show the product therein.

FIG. 3 is a plan view of one embodiment of a package of the present invention.

FIG. 4 is a plan view of an alternative embodiment of a package of the present invention.

FIG. 5 is a plan view of an alternative embodiment of a package of the present invention.

FIG. 6 is a plan view of an alternative embodiment of a package of the present invention with a portion cut away to show the contents of the package.

FIG. 7 is a plan view of an alternative embodiment of a package of the present invention with a portion cut away to show the contents of the package.

FIG. 8 is a plan view of one embodiment of the present invention showing a number of packages on a storage or display unit.

FIG. 9 is a plan view of an alternative embodiment of the present invention showing a number of packages on a storage or display unit.

FIG. 10 is a plan view of an embodiment of the present invention showing a number of packages on a storage or display unit.

FIG. 11 is a plan view of an embodiment of the present invention showing a number of packages on a storage or display unit.

FIG. 12 is a plan view of an array of packages on a storage or display unit.

### DETAILED DESCRIPTION OF THE INVENTION

As noted above, the present invention is directed to improved packages, arrays of packages and methods for advertising products. The following terms used herein have the following meanings.

“Array of packages”, as used herein, refers to an arrangement of at least two packages.

“Brand logo”, as used herein, refers to indicia associated with a particular product or product line. The brand logo may include any combination of words, symbols, pictures or other graphic or textual elements.

“Brand name”, as used herein, refers to a name for a product or product line. Generally, such brand names are trade names or trademarks, but can also include generic or descriptive language.

“Consumer”, as used herein, refers to any person that purchases, makes a decision to purchase a product or is contemplating making a purchase of a product. Typically, the Consumer will be the end user of the product, but the definition of consumer, as used herein is not so limited.

“Equity building advertising”, as used herein, refers to a message about one or more of the benefits of a product. The equity building advertising can include letters, text, words, graphics, symbols, pictures or any other indicia related to the benefits of a product.

“Package”, as used herein, refers to any covering material or wrapper in which at least a portion of a product is placed for shipping, storage and/or sale.

“Product”, as used herein, refers to any object, material or substance that is intended for use by a consumer.

“Promotional message”, as used herein, refers to a message that provides a consumer with information about a product other than the product on which the message is provided. At times, such promotional messages are not provided on the product or product packaging itself, but generally in close proximity to the product, such as, for example on the same store shelf or alternatively, provided via a separate medium, such as a coupon, a print, radio, internet or television advertisement or another form of a special offer, such as, for example, a prize, a give-away item, an offer to sell an item or items at a discount, etc. Such promotional messages differ from equity building advertising in that promotional messages do not provide information about one or more benefits of the product that includes the promotional message.

“SKU”, as used herein is a stock keeping unit. Stock keeping units are used to uniquely identify particular products. Generally, each product sold by a retailer will have a separate and unique SKU. Thus, all different sizes, forms, package counts, etc. will typically have a unique SKU. Often the SKU is provided as a series of numbers and/or a bar code and is often associated with the UPC of a product.

“UPC”, as used herein refers to a universal product code. UPCs are codes intended to help identify the manufacturer of a product. Accordingly, each manufacturer has a unique UPC. Often, UPCs are on packages in the form of a series of numbers and/or a bar code.

Package

As noted above, the present invention includes an improved package for products. As with most packages, the package of the present invention provides a means for storing, shipping, protecting, containing and/or displaying the product or products that are disposed within or partially within the package. Packages are also used to identify the product(s) therein and provide the consumer with general information about the product. The package of the present invention provides an improvement to prior art packaging in that it includes an equity building advertising message for the product that is disposed in the package. In certain embodiments, the package may include equity building advertising for products that are not in the particular package itself, but other products of similar nature (e.g. the same product in a different package, a similar product with a different aesthetic feature, sheet count or volume, products with the same brand name and/or products from the same manufacturer). In alternative embodiments, the package may include an equity building advertising message for products from a different manufacturer, having a different brand name, being dissimilar in nature and/or having no connection with the product in the package including the equity building advertising. Further still, the package may include an equity building advertising message that relates to the advantages of two or more products, including messages that describe the benefits of using the products together or otherwise in conjunction with each other.

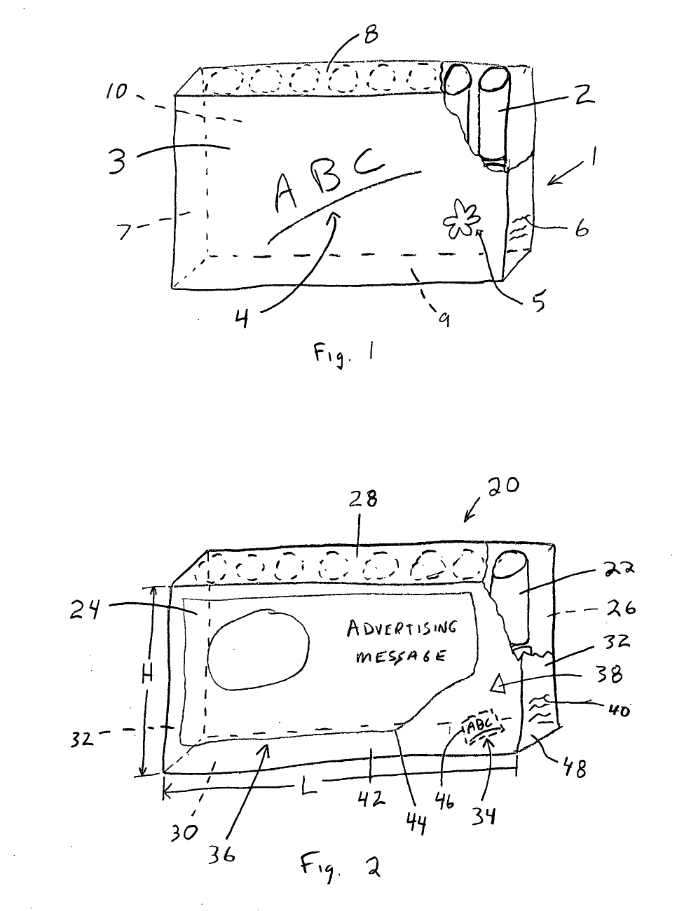

An example of a prior art package is shown in FIG. 1. A portion of the package 1 is cut away to show the products 2 that are disposed within the package 1. The package 1 includes a display panel 3 that is intended to be oriented such that the consumer can see the display panel 3 when the package 1 is displayed for sale. This particular package 1 also includes a brand name and/or brand logo 4. The brand name/brand logo 4 is prominent on the display panel 3 and is intended to help the consumer identify the particular product 2 within the package 1 and possibly the manufacturer or distributor of the product 2. The package 1 may also include decorative indicia 5 and/or technical or other written information 6 about the product 2, itself. Typically, the brand name/brand logo 4 is the most prominent indicia on the display panel 3 and any other decorative indicia 5 or information 6 is of lesser prominence and or provided on portions of the package other than the display panel 3, such as, for example, at least one side panel 7, a top panel 8, a bottom panel 9 or a back panel 10. (Of course, not all packages will be designed so as to include all of the panels of a parallelepiped, but generally packages for consumer products will include at least one display panel 3 and portions that are not generally intended to be displayed to the consumer while displayed for sale.)

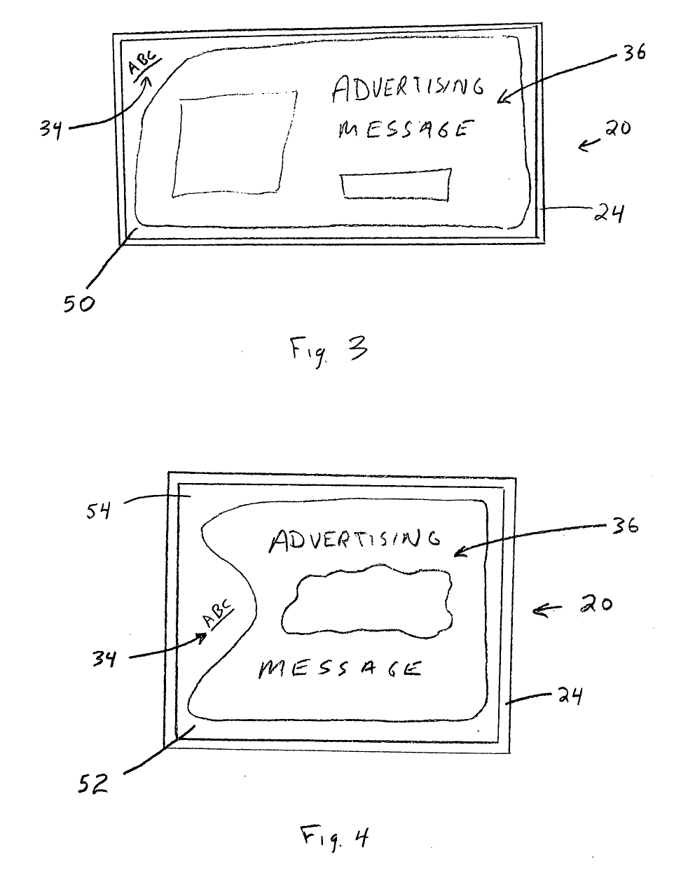

FIG. 2 is an example of one embodiment of the present invention. As shown, the package 20 has a cut away section showing the products 22 disposed within the package 20. The package 20 includes a display panel 24 that is intended to be oriented such that the consumer can see the display panel 24 when the package 20 is displayed for sale. The package 20 shown also includes a rear panel 26, a top panel 28, a bottom panel 30 and side panels 32. However, the package of the present invention need not be of any particular size, shape or geometry. In any case, the package 20 will have a display panel 24, as noted above, that is intended to be oriented such that the consumer can see the display panel 24 when the package 20 is displayed for sale. For packages with non-planar surfaces, the display panel 24 is that portion of the package 20 that the consumer sees when looking at the package 20 on display for sale without removing the package 20 from its display and under typical shopping conditions for the particular product involved. Thus, for certain consumer product packages 20, the display panel 24 will be that portion of the package 20 that is seen by the consumer while the package 20 is on a store shelf somewhere between the ground and about 10 feet above the ground and when the consumer is viewing the package 20 while located within about 25 feet of the package 20. (Examples of the package 20 being displayed on typical shelving of a retail store are shown, for example in FIGS. 3 and 4. However, the package 20 may be displayed in any suitable manner and normal viewing conditions may be different than those described above and/or shown in the Figures. Thus, the above description and the figures should not be construed as in any way limiting to the scope of the invention, but rather as examples of how a consumer might view the display panel 24 of a package 20.)

The particular package 20 shown in FIG. 2 includes a brand name and/or brand logo 34. The brand name/brand logo 34 is disposed on the display panel 24 and is intended to help the consumer identify the particular product 22 within the package 20 and possibly the manufacturer or distributor of the product 22. However, in some cases, the package 20 may not include any brand name/brand logo 34 on the display panel 24. The package 20 also includes an equity building advertising message 36 (and thus, may also be referred to herein as an “equity building package 20”). In certain embodiments, the package 20 may also include decorative indicia 38 and/or technical or other written information 40 about the product 22, itself or other information relevant to the end user of the product 22.

As shown in FIG. 2, the brand name/brand logo 34 is not the most prominent indicia on the display panel 24, but is of lesser prominence than the equity building advertising message 36. In fact, in this particular embodiment, the equity building advertising message 36 is of greater prominence than any other indicia on the display panel 24. In terms of prominence, there are a number of factors that can be involved with elevating or diminishing the prominence of a feature on a package, including, but not limited to the size of the indicia, the colors used, the texture, the area of coverage dedicated to the particular indicia versus other indicia, the location of the indicia on the package or display panel, the aesthetic appeal of the indicia versus other indicia or parts of the package and/or the message provided by the particular indicia.

In certain embodiments, as shown for example, in FIG. 2, display panel 24 will have a display panel surface area 42 determined by the length L of the display panel 24 of the package 20 and the height H of the display panel 24 of the package 20. (Of course, packages with other shapes will have different measurements that go into determining the overall display panel surface area 42, but in each case such measurements should be easily determined by one of skill in the art. For packages with non-planar display surfaces, the display panel is considered to be the portion of the package that the consumer is intended to see when the package is displayed for sale.)

As shown in the embodiment of the invention shown in FIG. 2, the package 20 of the present invention may include an equity building advertising message 36 that is more prominent than the brand name or brand logo 34 in terms of the area that the equity building advertising message 36 covers on the display panel 24 versus the area of the display panel 24 that the brand name or brand logo 34 covers. For example, it may be desirable for the area of the display panel 24 covered by the equity building advertising message 36 (hereinafter “equity area” 44) to be greater than the area of the display panel 24 covered by the brand name and/or brand logo 34 (hereinafter “brand name/logo area” 46). The equity area 44 is the entire area of the display panel 24 that is covered by indicia related to the equity building advertising message 36, including all text, graphics and background copy that relates to the equity building advertising message 36. In FIG. 2, the equity area 44 is the entire area encompassed by the line indicated by the number 44. The brand name/logo area 46 is the area of the display panel 24 that is covered by indicia related to the brand name and/or brand logo 34. In FIG. 2, the brand name/logo area 46 is shown by the dotted line surrounding the brand name/brand logo indicia 34.

In one example, the equity area 44 may be at least about 50% of the area of the display panel surface area 42. In other embodiments, the equity area 44 may be at least about 60%, at least about 70%, at least about 75%, at least about 85% or any percentage greater than about 50% of the display panel surface area 42. Likewise, it may be desirable for the brand name/logo area 46 be less than about 50%, less than about 40%, less than about 30%, less than about 25%, less than about 15% or any other percentage less than about 50% of the display panel surface area 42. It may also be desirable that the equity area 44, or the area of the equity building advertising message 36, is greater than the brand name/logo area 46. In alternative embodiments, it may be desirable that the equity area 44 be at least about twice as large as the brand name/logo area 46, at least about 3 times as large as the brand name/logo area 46, at least about five times as large as the brand name/logo area 46, at least about 10 times as large as the brand name/logo area 46, or greater.

The package 20 of the present invention may include more than one panel or face that has the equity building advertising message 36, as described above. In fact, it may be desirable in certain situations to provide the package 20 with more than one panel or face having generally the same look and indicia. For example, on a package like the one shown in FIG. 2, it may be desirable for the rear panel 26 to be identical to or nearly identical to the display panel 24. This can help ensure that the equity building advertising message 36 of the display panel 24 is displayed to the consumer even if the package 20 is purposefully or mistakenly oriented such that the display panel 24 is not facing the consumer when displayed for sale. It can also help ensure that the equity building advertising message 36 is displayed even if the package(s) 20 are not displayed in an orderly fashion, such as, for example, at the end of a shopping day when the packages 20 are not fully stocked and/or may have been re-oriented by shoppers. In other embodiments, any other panel(s) or face(s) of the package 20, such as, for example, the top panel 28, the bottom panel 30, and/or either or both of the side panels 32 may also include the same or similar equity building advertising message 36 as the display panel 24. The equity building advertising message 36 on such other panel(s) can be presented as described above (i.e. having the same or similar proportions of coverage of the panels as set forth above), or may be presented in a different manner or with different indicia.

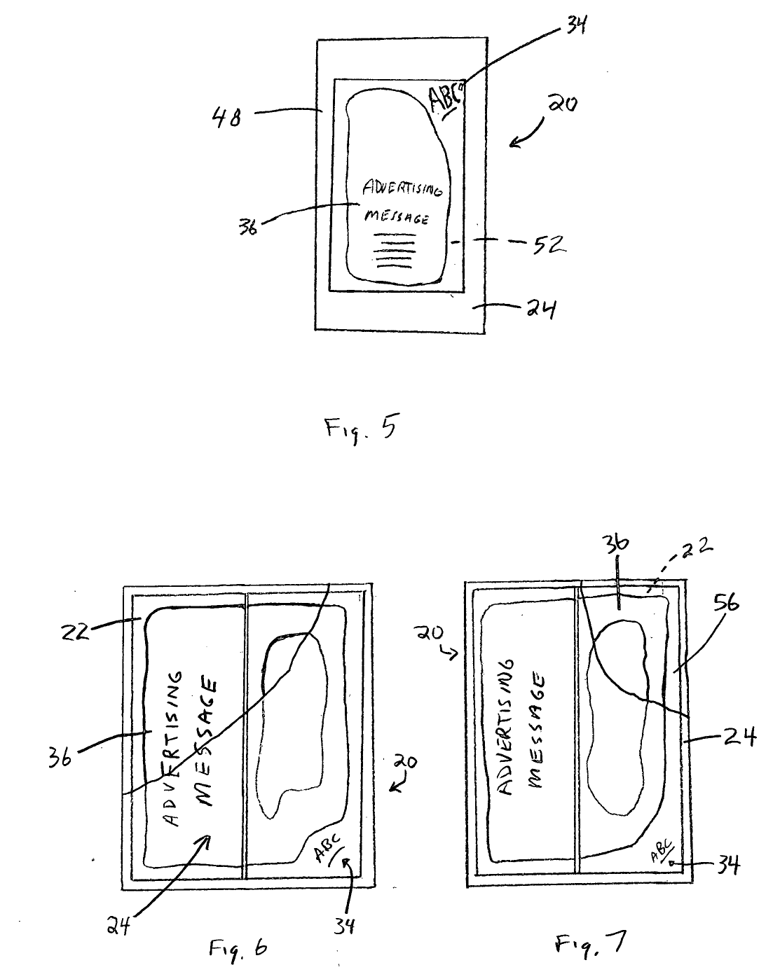

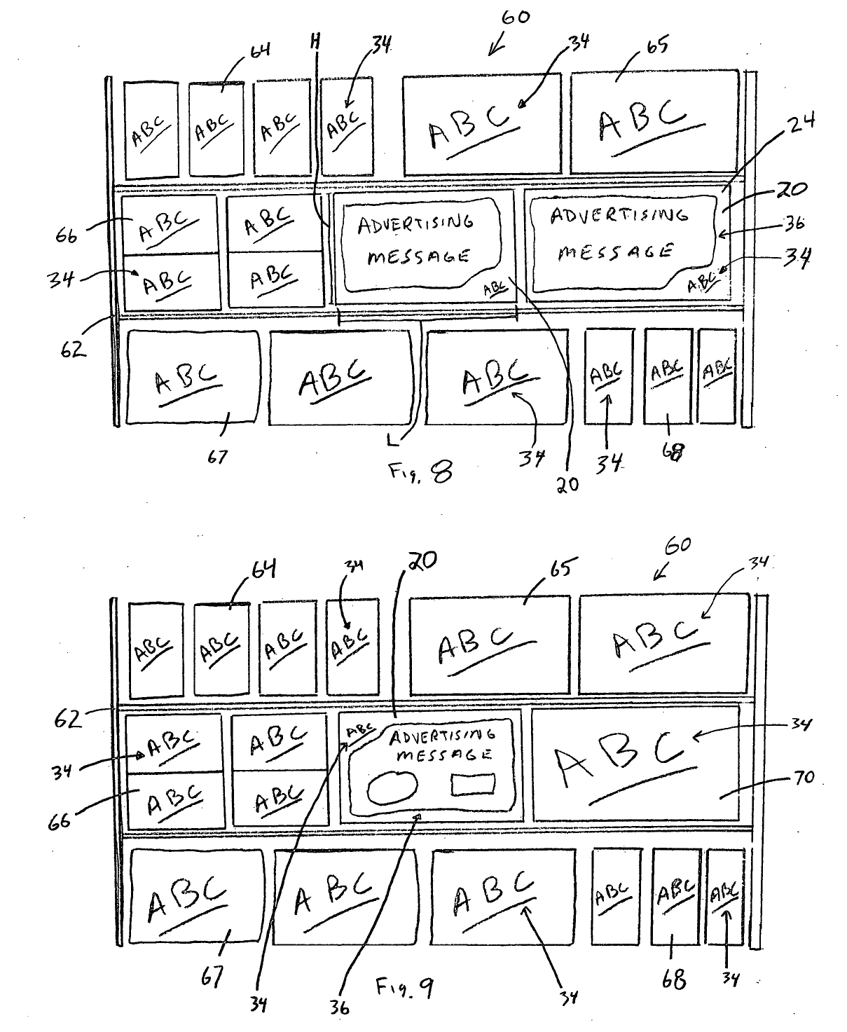

The equity building advertising message 36, the brand name and/or logo 34, any decorative indicia 38, written information 40 or any other advertising message, promotional message or other aesthetic indicia may be provided on the package 20 in any suitable way. For example, the display panel 24 of the package 20 may have the equity building advertising message 36 and any other indicia printed on the inner or outer surface of the material 48 making up the package 20. In other embodiments, the equity building advertising message 36 and/or any other indicia may be provided on a separate sticker or label 50 or material that is temporarily or permanently joined to the display panel 24 of the package 20 (one example of which is shown in FIG. 3). In other embodiments, the package 20 may be provided with an insert 52 that is provided within the package 20 and can be seen through an opening 54 in the package 20 (one example of which is shown in FIG. 4) or through the material 48 of the package 20 (one example of which is shown in FIG. 5). In yet other embodiments, the equity building advertising message 36 may be provided on one or more of the products 22 within the package 20 (one example of which is shown in FIG. 6) or on primary packaging 56 within which the product(s) are packaged and then over-wrapped by the package 20 (one example of which is shown in FIG. 7).

The package 20 shown in FIG. 2 represents a package of rolled paper products, such as paper towels or bath tissue. However, the package 20 of the present invention is in no way limited to a particular type or category of products. Thus, the package 20 can be practiced with any product therein. For example, the package 20 may include products such as facial tissue, bath tissue, towels, paper towels, hair care products, food products, laundry products, fabric care products, oral care products, pharmaceuticals, over the counter medications, feminine care products, diapers, incontinence products, anti-persperant/deodorant products, pet food, cosmetics, batteries, coffee, food storage containers, garbage bags, surface cleaning products, soap, wipes or other consumer products. Further, the package 20 of the present invention may include a single product or several products (e.g. a multi-pack of paper towels or bath tissue.) Further, the package 20 of the present invention may include a number of different products, either of the same general type or class (e.g. paper towels with different prints or roll sheet counts, etc.) or of related products (e.g. a tube of toothpaste packaged with a toothbrush) or products that are completely unrelated to each other (e.g. a package of tissue with a hand cleanser).

The package 20 of the present invention may be made of any suitable material and may be of any shape or size. Although it is desirable in certain instances that the package 20 has at least one generally planar face, typically the display panel 24, such should not be considered a requirement of the present invention. Further, there is no requirement that the package 20 have any particular number of sides. In fact, the package 20 may be cylindrical, conical, spherical or any other geometric shape or combination of shapes.

In some instances, it may be desirable for the package 20 to have a display panel 24 that is at least about 20 cm in length and a height of at least about 20 cm. In alternative embodiments, it may be desirable that the package 20 has a display panel 24 of at least about 30 cm, at least about 40 cm, at least about 50 cm or at least about 75 cm in length. Likewise, it may be desirable that the package 20 has a display panel 24 of at least about 30 cm, at least about 40 cm, at least about 50 cm or at least about 75 cm in length. Any combination of the above noted dimensions or any other dimensions may be used in the package 20 of the present invention. However, one advantage of the package 20 of the present invention is that it provides a medium for placing at least the equity building advertising message 36, and thus, larger display panels 24 can provide the package 20 with a billboard-like effect for displaying the equity building advertising message 36. This is especially impactful when the package(s) 20 are displayed in an array of packages or along side or surrounded by other packages.

Array of Packages

The present invention also includes an array of packages for storing and/or displaying products. One advantage of the array of packages of the present invention is that it provides a way to uniquely display the packages in a way that may help increase the visibility of the packages on display. The array of the present invention may also provide an improved means for attracting the attention of a potential consumer. Additionally, the array of packages of the present invention may provide an improved means for informing the consumer about the benefits of a particular product that may help build the product's brand equity. In yet other embodiments, the array of the present invention may provide a means for informing the consumer about the benefits of a particular product line that may help build the product line's brand equity.

As noted above, an array of packages, as used herein, refers to an arrangement of two or more packages. The array can include packages that are all the same in terms of size, shape, the product within the package, the package SKU or any other feature of the package, or may include packages of different sizes, shapes, products within the package, the package SKU or any other feature of the package. The packages may be arranged in a free-standing condition (i.e. standing and/or stacked on the ground or floor without any display structure holding them) or may displayed on a structure such as a shelf, pallet, hanger, in a cabinet, or any other structure designed to or used for storing and/or displaying the packages for sale. The array of packages can be located such that they are distant or removed from other products and/or packages or may be located next to or intermingled with other products or packages of the same or different manufacturer.

In one embodiment of the present invention, as shown in FIG. 8, for example, the array of packages 60 is disposed on a shelving unit 62. The individual packages of the array of packages 60 include packages 20, 64, 65, 66, 67 and 68. In the particular embodiment shown in FIG. 8, the array of packages 60 includes a number of different packages 20, 64, 65, 66, 67 and 68 having the same brand name and/or brand logo 34. In certain embodiments, the array of packages 60 includes different packages having the same or similar products therein. For example, the array of packages 60 shown in FIG. 8 could represent an array of packages 60 including paper products such as paper towels, bath tissue or facial tissue, although any other product or products could also be in the array 60. The different packages 20, 64, 65, 66, 67 and 68 could be different size packages, packages with different amounts (e.g. number of rolls or sheets) of product in the package, packages including products with different aesthetic, olfactory, structural or chemical features and/or packages with different SKUs and/or UPCs. Further, the array of packages 60 of the present invention may include a number of different packages including products that are not of the same type, but rather are related products (e.g. a tube of toothpaste packaged with a toothbrush) or products that are completely unrelated to each other (e.g. a package of tissue with a hand cleanser).

As shown in FIG. 8, the array of packages 60 includes at least package 20 including the equity building advertising message 36, as set forth above. In the particular array of packages 60 shown, there are two packages 20 that have the equity building advertising message 36, but any number of the packages in the array of packages 60 can include the message 36. Also, as shown, the packages 20 having the equity building advertising message 36 are designed such that the equity building advertising message 36 is disposed on the display panel 24 such that the equity building advertising message 36 can be seen by a consumer looking at the package 20, as it is displayed for sale.

The equity building package(s) 20 included in the array of packages 60 shown in FIGS. 8 and 9 have the characteristics of the packages 20 described above. That is, the brand name/brand logo 34 is not the most prominent indicia on the display panel 24, but is of lesser prominence than the equity building advertising message 36. In fact, in the embodiments shown in FIGS. 8 and 9, the equity building advertising message 36 is of greater prominence than any other indicia on the display panel 24. As noted above, in terms of prominence, there are a number of factors that can be involved with elevating or diminishing the prominence of a feature on a package, including, but not limited to the size of the indicia, the colors used, the texture, the area of coverage dedicated to the particular indicia versus other indicia, the location of the indicia on the package or display panel, the aesthetic appeal of the indicia versus other indicia or parts of the package and/or the message provided by the particular indicia.

As noted above, the equity building package(s) 20 of the present invention may include an equity building advertising message 36 that is more prominent than the brand name or brand logo 34 in terms of the area that the equity building advertising message 36 covers on the display panel 24 versus the area of the display panel 24 that the brand name or brand logo 34 covers. For example, it may be desirable for the equity area 44 (the area of the equity building advertising message 36) to be greater than the brand name and/or brand logo area 46. In one example, the equity area 44 may be at least about 50% of the area of the display panel surface area 42. In other embodiments, the equity area 44 may be at least about 60%, at least about 70%, at least about 75%, at least about 85% or any percentage greater than about 50% of the display panel surface area 42. Likewise, it may be desirable for the brand name/logo area 46 be less than about 50%, less than about 40%, less than about 30%, less than about 25%, less than about 15% or any other percentage less than about 50% of the display panel surface area 42. In alternative embodiments, it may be desirable that the equity area 44 be at least about twice as large as the brand name/logo area 46, at least about 3 times as large as the brand name/logo area 46, at least about five times as large as the brand name/logo area 46, at least about 10 times as large as the brand name/logo area 46, or greater.

As with the packages described above, the equity building package(s) 20 of the present invention may include more than one panel or face that has the equity building advertising message 36. In fact, as noted above, it may be desirable in certain situations to provide the equity building package 20 with more than one panel or face having generally the same look and indicia. For example, on equity building packages 20 like those shown in FIGS. 8 and 9, it may be desirable for the rear panel 26 to be identical to or nearly identical to the display panel 24. This can help ensure that the equity building advertising message 36 of the display panel 24 is displayed to the consumer even if the equity building package 20 is purposefully or mistakenly oriented such that the display panel 24 is not facing the consumer when displayed for sale. It can also help ensure that the equity building advertising message 36 is displayed even if the equity building package(s) 20 are not displayed in an orderly fashion, such as, for example, at the end of a shopping day when the display shelves 62 are not fully stocked and/or the packages may have been re-oriented by shoppers. As with the packages described above, the equity building package(s) 20 may have other panel(s) or face(s), such as, for example, the top panel 28, the bottom panel 30, and/or either or both of the side panels 32 that also include the same or similar equity building advertising message 36 as the display panel 24.

The equity building advertising message 36, the brand name and/or logo 34, any decorative indicia 38, written information 40 or any other advertising message, promotional message or other aesthetic indicia may be provided on the equity building package(s) 20 in any suitable way, such as, for example, the ways and means described above with respect to the package 20 shown in FIG. 2. Further, as noted above, the package(s) 20 of the present invention or any other packages of the array of packages 60 may be made of any suitable material and may be of any shape and/or size. Also, any of the packages in the array 60 may have display panels 24 that include, but are not limited to those dimensions set forth above.

As noted above, the array of packages 60 shown in FIGS. 8 and 9 are representative of arrays of packages that include a number of different packages having the same brand name/brand logo 34. The arrays 60 include packages of different sizes. The different sized packages may include products of different sizes or different numbers of products in each package. For example, if the products are rolled paper products, the packages may all include a single brand or line-up of bath tissue or paper towel, but may include different roll counts, roll sizes, substrates, plies, printing on the product or color of the product, scents, price, indicia on the product or package, usage instructions or other differences. Generally, such different packages will have different SKU numbers and/or different UPCs.

In the array of packages 60 shown in FIG. 8, the array 60 includes packages 20, 64, 65, 66, 67 and 68. Packages 20 include the equity building advertising message 36. The size and make-up of the equity building advertising message 36 on the packages 20 can be as set forth above. For example, at least the display panel 24 of the packages 20 may include an equity building advertising message 36 that is more prominent than the brand name/brand logo 34 on the display panel 24 of the packages 20, as described herein. The rest of the packages, packages 64, 65, 66, 67 and 68 do not include the equity building advertising message 36. However, such packages could include the equity building advertising message 36 as well as any other promotional, instructional or aesthetic features desired. In the particular embodiment shown, the packages 20 including the equity building advertising message 36 or the equity building packages 20 are located on the display shelf 62 such that at least one of the equity building packages 20 is adjacent at least one package from the same product line-up (e.g. having the same brand name/brand logo 34) with graphics or indicia that are different than the graphics and/or indicia on the equity building packages 20. In an alternative embodiment, the array of packages could be such that the equity building package(s) 20 are separated from the other packages of the same product line-up, but still within view of such other packages. In yet other embodiments, one or more of the equity building packages 20 could be adjacent packages from the same product line-up on at least two sides, at least three sides or completely surrounded by such other products.

In the embodiment of the present invention shown in FIG. 9, the array of packages 60 includes at least one equity building package 20 and at least one package 70 that is the same in all respects to the equity building package, but does not include the equity building advertising message 36 on the display panel 24. Thus, for example, the packages 20 and 70 would be the same SKU and include the same products, but the equity building package 20 would include the equity building advertising message 36 and the package 70 would not, at least on the display panel 24. In other embodiments, the array of packages could include the equity building package 20 and the package 70 which is similar in all respects, as noted above, but the package 70 does include the equity building advertising message 36 on the display panel 24. However, in such embodiments, the equity building advertising message 36 on the display panel 24 is of lesser prominence than the equity building advertising message 36 on the display panel of the equity building package 20. Of course, the array of packages 60 in any case could include any number of other packages, such as packages 64, 65, 66, 67 and 68. As shown, such packages include the same brand name/brand logo 34 as the equity building package(s) 20, but are different in some way, for example, different sizes, substrates, plies, printing on the product or color of the product, scents, price, indicia on the product or package, usage instructions or other differences. Typically, such different packages would have different SKU numbers and/or different UPCs.

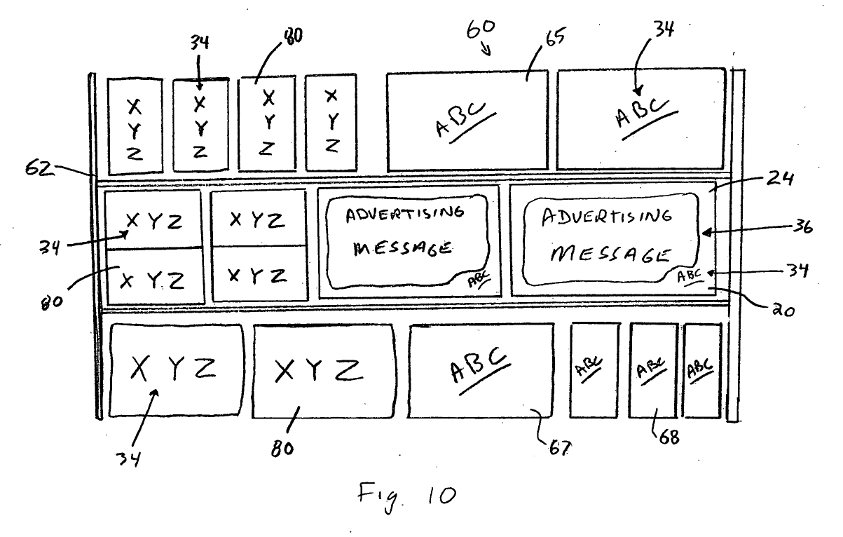

The array of packages 60 of the present invention may be provided as a display of packages separate from other packages not in the array or may be located within a display containing packages other than those in the array of packages 60 of the present invention, as shown, for example, in FIG. 10. In FIG. 10, the array of packages 60 of the present invention is located on a display, shelves 62, with other packages 80 that are not part of the array 60. In the particular embodiment shown, the packages 80 that are not part of the array of packages 60 of the present invention include packages having a different brand name/brand logo 34 than the brand name/brand logo 34 of the packages 20, 65, 67 and 68 of the array of the packages 60 of the present invention. In the particular shelf setup shown in FIG. 10, the packages 80 represent packages of product that are similar in nature to the packages 20, 65, 67 and 68 of the array of packages 60 of the present invention. Thus, for example, the packages 80 could be another manufacturer's brand of paper towels if the array of packages 60 included packages of paper towels. Alternatively, the packages 80 could include any number of different products, including products of the same general nature, i.e. paper products but not paper towels, or could be completely different products from the products in the packages 20, 65, 67 and 68 of the array of packages 60 shown. Further, unlike what is shown in FIG. 10, the packages 80 that are not part of the array of packages 60 of the present invention could be any number of different brands and FIG. 10 should not be construed to limit the particular arrangement of the array of packages 60 of the present invention or the surrounding packages 80. Rather, the figure is just one exemplary embodiment used to help illustrate the impact of the equity building package 20 and the array of packages 60 including the equity building package 20.

Method of Displaying an Array of Packages

The present invention also includes a method of displaying an array of packages including one or more products therein. In particular, the method includes displaying an array of packages in a retail or wholesale setting. The method includes providing an array of packages including at least one first equity building package having a first display panel including certain indicia, wherein the first display panel is intended to be displayed to the consumer when the package is displayed for sale. The method also includes providing at least one second package that includes indicia that are different than the indicia on the display panel of the equity building package.

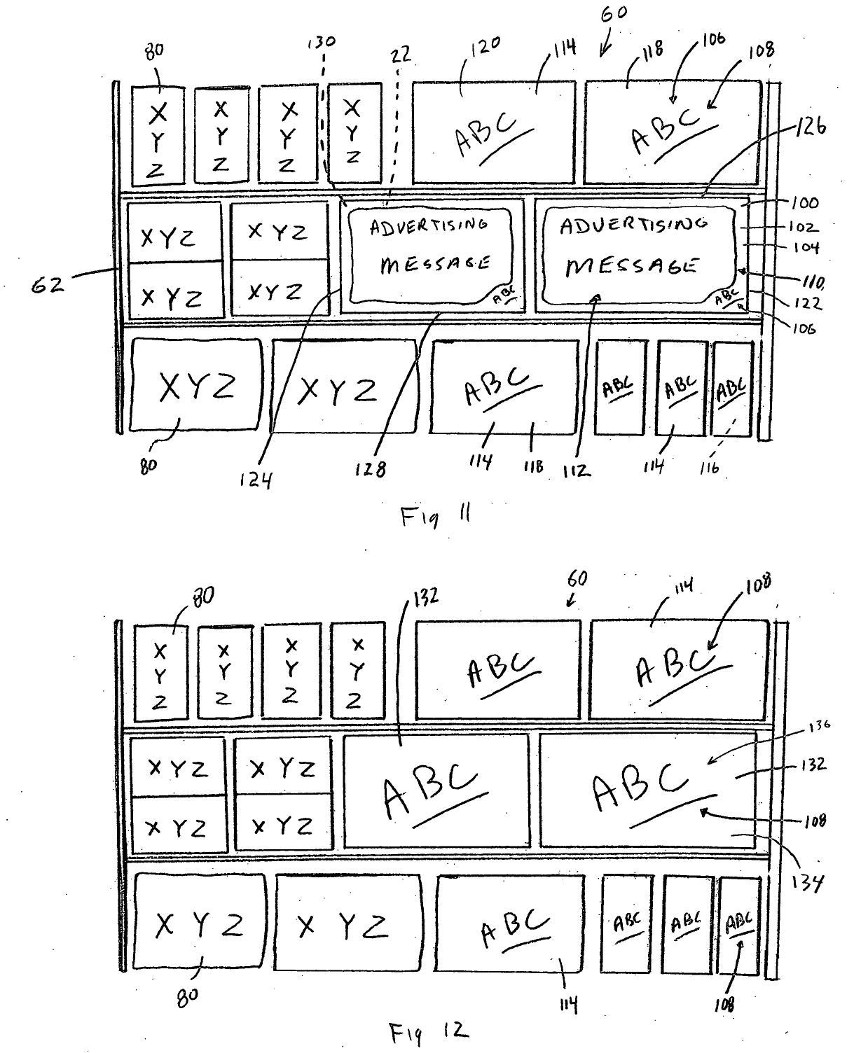

In one embodiment, as shown in FIG. 11, the method for displaying an array of packages 60 includes providing at least one first package 100, such as one of the package(s) 20 of FIGS. 2-10, including at least one product 22 therein. The first package 100 has at least a first display face 102. The first display face 102 has a first display area 104 and includes first indicia 106 representing brand name/brand logo 108 of the product 22 and or the manufacturer of the product 22. The first package 100 also includes second indicia 110 including an equity building advertising message 112. The second indicia 110 of the first package 100 cover more of the area 104 of the first display face 102 than the first indicia 106. The method also includes providing at least one second package 114 (such as, for example one or more of packages 64, 65, 66, 67, 68 and/or 70 as shown in FIGS. 8 and 9). The second package 114 includes at least one product 116 therein and has a second display face 118. The second display face 118 has a second display face area 120 and includes the first indicia 106 of the first package 100.

In one embodiment of the present invention, the array of packages 60 includes a first package 100 including a first display face 102 that includes first indicia 106, as set forth above and second indicia 110 that does not include the equity building advertising message 112. In another embodiment, the second display face 118 second package 114 does not include the equity building advertising message 112.

The method of the present invention may also includes locating at least one first package 100 close enough to at least one second package 114 such that both the first package 100 and the second package 114 can both be seen by the consumer at the same time. In certain embodiments, it may be desirable that at least one first package 100 is located adjacent at least one second package 114, as is shown in FIG. 11, but the packages 100 and 114 need not be adjacent each other in all embodiments. It may also be desirable to provide an array of packages 60 wherein at least one first package 100 is located such that at least two sides of the first package 100 are adjacent one or more second packages 114. This too is shown in FIG. 11, wherein the first package 100 has a right side 122, a left side 124, a top side 126, a bottom side 128 and a back side 130.

In one embodiment of the present invention, an array of packages 60 is provided, as stated above, including at least one first package 100 and at least one second package 114. In this embodiment, the first package 100 has a first SKU. The second package 114 that is provided has a second SKU that is different from the first SKU. In certain embodiments, the first product 22 in the first package 100 may be the same as the second product 116 in the second package 114. In such cases, the first package 100 and the second package 114 with different SKUs will generally differ in at least one way. For example, the first package 100 and the second package 114 may have one or more of the following differences: product size or number or products in the package, amount of material on the product, indicia or graphics on the product, color of the product, product usage instructions or the like. In other embodiments, the first product 22 in the first package 100 may be physically or chemically different in some way from the second product 116 in the second package 114. For example, the first product 22 could be a base or standard quality product and the second product could be a premium or economy product.

In yet another embodiment of the present invention, a method of rotating an array of packages 60 is provided. The method includes providing at least one first package 100, such as one of the package(s) 20 of FIGS. 2-10, including at least one product 22 therein. The first package 100 has at. least a first display face 102. The first display face 102 has a first display area 104 and includes first indicia 106 representing brand name/brand logo 108 of the product 22 and or the manufacturer of the product 22. The first package 100 also includes second indicia 110 including an equity building advertising message 112. The second indicia 110 of the first package 100 cover more of the area 104 of the first display face 102 than the first indicia 106. The method also includes providing at least one second package 114 (such as, for example one or more of packages 64, 65, 66, 67, 68 and/or 70 as shown in FIGS. 8 and 9). The second package 114 includes at least one product 116 therein and has a second display face 118. The second display face 118 has a second display face area 120 and includes the first indicia 106 of the first package 100. The method also includes replacing at least one of the first packages 100 with a third package 132 (as shown in FIG. 12). The third package 132 includes a third display panel 134 having an area and including third indicia 136 that includes at least the brand name/brand logo 108 of the first package 100. In certain embodiments, the third indicia 136 may also include the first indicia 106 of the first package 100. However, in such embodiments, the first indicia 106 covers at least as much area of the third display panel 134 as the second indicia 110.

In certain embodiments of the present invention, the method of rotation of the array of packages 60 includes replacing at least one first package 100 with at least one third package 132 that includes third indicia 136 that are substantially the same as the second indicia 110 of the second package(s) 114. That is, the third package(s) 132 and the second package(s) will all have a similar look, such as is shown in FIG. 12. The array of packages 60, as shown in FIG. 12, where all of the packages of a particular product line are similar, is what is commonly used in retail settings today.

It may also be desirable to provide an array of packages 60 with similar indicia across the line-up, such as the line-up shown in FIG. 12 prior to and/or after providing the first package 100 in the array. (Such an array could include a “standard” package having a display panel including standard indicia thereon, wherein the standard indicia are substantially similar to the third indicia.) Thus, the first package(s) 100 may be provided only temporarily in the array of packages 60. In one embodiment, the first packages 100 are replaced with the third packages 132 as the first packages 100 are purchased by the consumer. Thus, where a number of first packages 100 are displayed at one time, the replacement of the first packages 100 with the third packages 132 will take place over some period of time, typically greater than 24 hours. In other embodiments, the first packages 100 are all replaced with the third packages 132 over a period of time shorter than 24 hours. This would likely be the case if the array of packages 60 were rotated during a single re-stocking or other similar event. In any case, it is contemplated that the method of the present invention could be implemented without having to change the normal distribution system for the packages of product in the array 60. That is, the packages could be shipped in the normal course of business and there would be no need for any type of business interruption or special equipment to handle the method of rotating the array of packages 60.

Such a method of rotating the first package(s) 100 in and out of the array of packages 60 may provide the array of packages 60 with improved visibility to the consumer and even an increase in purchase intent. This can be due not only to the change in the packaging in the array of packages 60 that makes the packages in the array of packages 60 stand out from other packages or arrays of packages, but also may be due to the equity building advertising message 112 that is provided on the first package(s) 100. The equity building advertising message 112 that is provided on the first package(s) 100 can help provide increased visibility of the first package(s) 100 as well as the entire array of packages 60 and thus, can provide a means for increasing purchase intent for the product in the first package 100 as well as the products in any of the other packages in the array 60. As such, in certain embodiments, the second packages 114 may benefit from or rely on the equity building advertising message 112 of the first package 100 to increase the equity in the product in the second package 114. Likewise, in certain embodiments, the first packages 100 may benefit from or rely on the brand name/brand logo 108 of the second package 114 to provide an indication of the brand of the product in the first package 100.

As noted above, the first indicia may include a trade name or trademark. Further, the equity building advertising message 112 may include information about the benefits of the product in the package itself, the packages in the array of packages 60 or any other product. Also, the equity building advertising message 112 may include at least one graphic element, at least one photographic image, text or any combination thereof. In certain embodiments, the equity building advertising message 112 that is provided on the first package 100 may include a graphic that includes a representation of the product in the package 100, and possibly even a representation of the product in use.

Further, all of the other characteristics and embodiments of the package 20 and the array of packages 60 described above in the sections labeled “Package” and “Array of Packages” are applicable to the methods of displaying the array of packages described in this section and the disclosure in those sections of the specification should be considered equally applicable to this section and as if it were repeated in detail in this section.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for displaying an array of packages, the method comprising:
providing at least one first package including a product therein, the first package having at least a first display face and a first non-display face, the first display face intended to be displayed to a consumer when the package is displayed for sale, the first display face having a first display face area and including first indicia having a first area and representing the name of the product or the product manufacturer and second indicia having a second area including an equity building advertising message for the product in the package, wherein the second area of the second indicia is greater than the first area of the first indicia on the first display face of the first package; and
providing at least a second package including a product therein, the second package having a second display face, the second display face intended to be displayed to a consumer when the package is displayed for sale, the second display face including the first indicia of the first package and not including the second indicia of the first package.

2. The method of claim 1 wherein the first package and the second package are provided in a location close enough to each other that, while being displayed, a consumer can see both the first package and the second package.

3. The method of claim 1 wherein the step of providing the first package includes locating the first package adjacent the second package.

4. The method of claim 1 where the first package has four sides, a right side, a left side, a top and a bottom, and wherein the step of providing the second package includes providing at least one second package adjacent at least two of the four sides of the first package.

5. The method of claim 1 wherein the first indicia is trademark or trade name.

6. The method of claim 1 wherein the second indicia does not include the name of the product or the product manufacturer.

7. The method of claim 1 wherein the second indicia of the first package includes an equity building advertising message that provides information about the benefits of the product in the package.

8. The method of claim 7 wherein the equity building advertising message includes at least one graphic element.

9. The method of claim 8 wherein the graphic element includes a photographic image.

10. The method of claim 8 wherein the graphic element includes a representation of the product included in the package.

11. The method of claim 8 wherein the graphic element includes a representation of the product included in the package in use.

12. The method of claim 11 wherein the second indicia further includes at some text.

13. The method of claim 12 wherein the text and the graphic element together provide information to the consumer in an attempt to get the consumer to purchase the product.

14. The method of claim 1 wherein the first area of the first indicia of the first package covers less than 40% of the first display face area of the first package.

15. The method of claim 1 wherein the first area of the first indicia of the first package covers less than 25% of the first display face area of the first package.

16. The method of claim 1 wherein the first area of the first indicia of the first package covers less than 15% of the first display face area of the first package.

17. The method of claim 1 wherein the first non-display face includes the first and second indicia of the first display face.

18. The method of claim 1 wherein at least the first package includes more than one product.

19. The method claim 18 wherein at least the first package is provided as an outer package and the products are provided in a primary package within the outer package that is distinct from the outer package.

20. The method of claim 19 wherein the products included in the outer package are rolled or stacked paper products.

21. The method of claim 1 wherein the product is selected from the group of facial tissue, bath tissue, towels, paper towels, hair care products, food products, laundry products, fabric care products, oral care products, pharmaceuticals, over the counter medications, feminine care products, diapers, incontinence products, anti-persperant/deodorant products, pet food, cosmetics, batteries, coffee, food storage containers, garbage bags, surface cleaning products, soap, wipes or other consumer products.

22. A method of presenting an array of packages, the method comprising:
providing at least a first package having an SKU and including a product therein, the first package having at least a first display face and a first non-display face, the first display face intended to be displayed to a consumer when the package is displayed for sale, the first display face having a first display face area and including first indicia having a first area and representing the name of the product or the product manufacturer and second indicia having a second area including an equity building advertising message for the product in the package, wherein the second area of the second indicia is greater than the first area of the first indicia on the first display face of the package; and
providing at least a second package having a different SKU than the first package and also including a product therein, the second package having a second display face, the second display face intended to be displayed to a consumer when the package is displayed for sale, the second display face including the first indicia of the first package, and wherein the second display face does not include the second indicia of the first package.

23. The method of claim 22 wherein the second indicia does not include the name of the product or the product manufacturer.

24. The method of claim 23 wherein the step of providing a first package includes providing at least one first product from a line-up of several different products including the same first indicia and the step of providing a second package includes providing a second package including at least one second product from the line-up, wherein the second product is different from the first product.

25. The method of claim 24 wherein the first product and the second product differ in at least one feature selected from the following group: product size, number of products in the package, amount of material in the product, price of the product, size of the package, indicia on the product or package, color of product, graphics on the product, or usage instructions on the product.

26. A method for rotating an array of packages, the method comprising:
providing at a first period of time an array of packages that can be seen by a consumer, the array of packages including:
at least one first package including a product therein, the first package having at least a first display face and a first non-display face, the first display face intended to be displayed to a consumer when the package is displayed for sale, the first display face having a first display face area and including first indicia having a first area and representing the name of the product or the product manufacturer and second indicia having a second area including an equity building advertising message for the product in the package, wherein the second area of the second indicia is greater than the first area of the first indicia on the first display face of the first package; and
at least one second package including a product therein, the second package having a second display face, the second display face intended to be displayed to a consumer when the package is displayed for sale, the second display face including the first indicia of the first package, and wherein the second display face does not include the second indicia of the first package; and
replacing at least one first package in the array of packages with at least one third package having a third display panel including third indicia that are different from the first and second indicia of the first package.

26. The method of claim 25 wherein the step of providing at least one first package includes providing the first package in a location where the consumer can see both the first package and the second package.

27. The method of claim 25 wherein the step of providing a first package includes providing at least one first package from a line-up of several different products including the same first indicia and the step of providing a second package includes providing a second package including at least one second product from the line-up, wherein the second product is different from the first product.

28. The method of claim 27 wherein the first product and the second product differ in at least one feature selected from the following group: product size, number of products in the package, amount of material in the product, price of the product, size of the package, indicia on the product or package, color of product, graphics on the product, or usage instructions on the product.

29. The method of claim 27 wherein the step of providing a third package includes providing a package including products that are the same as the products in the first package.

30. The method of claim 25 wherein the step of providing at least one first package includes providing a first package with a first SKU, the step of providing a second package included providing a second package with a second SKU and the step of replacing the first package with the third package includes replacing the first package with a first SKU with a third package with the first SKU.

31. The method of claim 25 wherein the step of replacing at least one of the first packages with at least one third package takes place after the at least one first package is sold to the consumer.

32. The method of claim 25 wherein the step of replacing at least one of the first packages with at least one third package includes replacing all of the first packages with one or more third package and such replacement takes place in less than about 24 hours.

33. The method of claim 25 wherein the step of replacing at least one of the first packages with at least one third package includes replacing all of the first packages with one or more third package and such replacement takes place over a time period of about 24 hours or more.

34. The method of claim 25 wherein the step of providing the first package includes locating the first package adjacent the second package.

35. The method of claim 25 where the first package has four sides, a right side, a left side, a top and a bottom, and wherein the step of providing the second package includes providing at least one second package adjacent at least two of the four sides of the first package.

36. The method of claim 25 wherein the second indicia does not include the name of the product or the product manufacturer.

37. The method of claim 25 wherein the equity building advertising message includes at least one graphic element.

38. The method of claim 37 wherein the graphic element includes a photographic image.

39. The method of claim 38 wherein the graphic element includes a representation of the product included in the package.

40. The method of claim 39 wherein the graphic element includes a representation of the product included in the package in use.

41. The method of claim 25 wherein the second indicia further includes at least some text.

42. The method of claim 41 wherein the text and the graphic element together provide information to the consumer in an attempt to get the consumer to purchase the product.

43. The method of claim 25 wherein the third indicia does not include the second indicia of the first package.

44. The method of claim 25 wherein the third indicia is different from the first and second indicia of the first package in that the third indicia includes the first indicia and the second indicia, but the second area of the second indicia is less than the first area of the first indicia.

45. The method of claim 25 wherein prior to providing the at least one first package, a standard package having a standard display panel is provided in the array of packages and is subsequently replaced by the first package, wherein the standard package has standard indicia on the standard display panel that are substantially identical to the third indicia on the third display panel of the third package.
